(19) United States
(12) Patent Application Publication
(10) Pub. No.: US 2026/0226458 A1
(43) Pub. Date: Aug. 6, 2026

(54) MULTI-SITE DUAL-FUNCTIONAL BASE EDITOR AND USE THEREOF

(71) Applicant: JIANGNAN UNIVERSITY, Wuxi (CN)

(72) Inventors: Long LIU, Wuxi (CN); Jian CHEN, Wuxi (CN); Xueqin LV, Wuxi (CN); Guocheng DU, Wuxi (CN); Jianghua LI, Wuxi (CN); Yanfeng LIU, Wuxi (CN); Yaokang WU, Wuxi (CN); Yang LI, Wuxi (CN)

(21) Appl. No.: 19/397,941

(22) Filed: Nov. 23, 2025

Related U.S. Application Data
(63) Continuation of application No. PCT/CN2023/140288, filed on Dec. 20, 2023.

(30) Foreign Application Priority Data
Jun. 14, 2023 (CN) 202310702843.5

(51) Int. Cl. C12N 15/11; C12N 9/22; C12N 9/78; C12N 15/70; C12N 15/75; C12N 15/90; C12R 1/125; C12R 1/19

(52) U.S. Cl. CPC C12N 15/111; C12N 9/226; C12N 9/78; C12N 15/70; C12N 15/75; C12N 15/90; C07K 2319/80; C12N 2310/20; C12R 2001/125; C12R 2001/19; C12Y 305/04001; C12Y 305/04002

(57) ABSTRACT

The invention provides a multi-site dual-function base editor and use thereof. Dual editing of multiple sites under the guidance of a single crRNA array is realized by using a constructed single-plasmid editor backbone, through the composition and structure optimization of a fusion protein formed of a cytosine deaminase, UGI, an adenine deaminase, and dCas12a. On this basis, the multi-site editing efficiency is further improved through the promoter replacement, the introduction and optimization of a synthetic spacing sequence in the crRNA array and the modification of a DR motif. Finally, the function of the multi-site dual-function base editor is verified through the generation of a multi-resistance strain, the de novo generation of a riboflavin producing strain, and the increased yield of a surfactin producing strain. According to the invention, the operable range of the dual-function base editor on the genome is widened.

### FIELD OF THE INVENTION

The invention relates to the field of biotechnologies, and discloses a multi-site dual-function base editor and use thereof.

### DESCRIPTION OF THE RELATED ART

Gene editing is a technical means to achieve gene knockout, foreign DNA fragment insertion or DNA base mutation by introducing a sequence change at a specific site on DNA. In recent years, the clustered regularly interspaced short palindromic repeat (CRISPR)/CRISPR-associated protein (Cas) system, particularly, CRISPR/Cas9 system, is widely used in gene editing. In this system, the immature precursor CRISPR RNA (pre-crRNA) can bind to trans-activating crRNA (tracrRNA), to form a crRNA:tracrRNA complex under the action of RNase III, which guides the Cas9 protein to recognize and cleave a specific site on the genome to produce double-strand breaks. Then, a sequence change on a homologous template is introduced to a target site on the genome through homology-directed repair (HDR), or the broken DNA fragments are directly connected by non-homologous end joining (NHEJ), and random insertion or deletion (Indel) occurs. Because the unrepaired double-strand breaks are fatal, only cells that are successfully repaired and introduced with mutations to avoid the recognition and cleavage by Cas9 can survive. This is the basic principle underlying gene editing using CRISPR/Cas9.

To simplify the operation process and improve the editing efficiency, crRNA and tracrRNA are often constructed into a chimera, that is, sgRNA (small guide RNA) for expression, so that gene editing can be carried out only by expressing sgRNA and Cas9 protein. At present, besides the CRISPR/Cas9 system, the CRISPR/Cas12a system (also known as CRISPR/Cpf1) is often used for gene editing. Unlike the CRISPR/Cas9 system, CRISPR/Cas12a functions only requiring crRNA. Moreover, Cas12a itself has the activity of RNase, and can cleave immature mRNA sequences containing multiple crRNAs. Therefore, a crRNA array containing multiple CRRNAs can be designed. When it is processed by Cas12a, multiple crRNAs with independent functions can be produced and guide Cas12a to target corresponding target sites on the genome, so as to realize the simultaneous editing of multiple sites.

Many species lack the NHEJ pathway (even if it is active, it is weak), the HDR pathway functions requiring the participation of a homologous template, and there will be competition between the two repair mechanisms, causing high mortality and low editing efficiency in the gene editing process based on the above methods. After the DNase of Cas9 is deactivated, nCas9 which can only cleave one DNA strand and dCas9 which can't cleave DNA are obtained. Although nCas9 and dCas9 can still bind to specific sites on the genome under the guidance of sgRNA, they will not cause fatal double-strand breaks. Similarly, after the DNase of Cas12a is deactivated, dCas12a which can't cleave DNA is obtained. Also, dCas12a can bind to a specific site on the genome under the guidance of crRNA without causing fatal double-strand breaks. In addition, because the RNase activity of dCas12a is retained, it can target many different sites on the genome under the guidance of a single crRNA array.

Cytosine deaminase can catalyze the deamination of cytosine (C) into uracil (U), and further convert U into thymine (T) through DNA repair or replication. The adenine deaminase TadA and a mutant thereof can deaminate adenine (A) into inosine (I), inosine will be read and copied as guanine (G) at a DNA level, to finally realize the transformation of A→G. As shown in FIG. 1a, the cytosine deaminase is fused with nCas9 or dCas9 to construct a cytosine base editor (CBE), which can realize genome base editing (C→T) independent of double strand breaks under the guidance of sgRNA. A uracil glycosylase inhibitor (UGI) derived from bacteriophage PBS is further fused to inhibit the activity of intracellular uracil DNA N-glycosylase (UNG), and prevent the base excision repair (BER) pathway from restoring U⋅G repair generated by editing into C⋅G pairing, thereby further improving the efficiency of base editing. As shown in FIG. 1b, the adenine deaminase is fused with nCas9 or dCas9 to construct an adenine base editor (ABE), which can realize A→G transformation at a specific site on the genome under the guidance of sgRNA. In addition, the cytosine deaminase and the adenine deaminase are both fused with nCas9 or dCas9 to construct a dual-function base editor (duBE), which can realize C→T and A→G transformations at specific sites on the genome under the guidance of sgRNA.

The site identified by Cas9 needs a PAM sequence with NGG (N=A, T, C, G), which limits the operational range on the genome of the dual-function base editor based on Cas9. In addition, every sgRNA that guides Cas9 needs a complete transcription unit, and several corresponding sgRNA expression frames need to be constructed during multi-site base editing (C→T and A→G), which not only increases the complexity of the construction process, but also reduces the stability of the DNA sequence due to the repeated use of the promoter and other elements. The dual-function base editor based on CRISPR/Cas9 has limited operational range on the genome, and low efficiency and complicated operations in the process of multi-site base editing (C→T and A→G). CBE or ABE based on dCas12a has been successfully constructed, and the PAM sequence is TTV (V=A, C, G), thus expanding the editable range on the genome. However, there are no reports on how to couple the cytosine deaminase, the adenine deaminase and dCas12a while their activities (C→T, A→G, crRNA array processing, and DNA targeting) are retained, to realize multi-site dual-function base editing (C→T and A→G) mediated by CRISPR/Cas12a.

### SUMMARY OF THE INVENTION

To solve the above problems, a multi-site dual-function base editor (MultiduBE) based on dCas12a is developed in the invention, by which multiple sites on the genome can be efficiently edited at the same time under the guidance of a single crRNA array (C→T and A→G). Dual editing (C→T and A→G) of multiple sites under the guidance of a single crRNA array is realized by using a constructed single-plasmid editor backbone, through the composition and structure optimization of a fusion protein formed of a cytosine deaminase, UGI, an adenine deaminase, and dCas12a. On this basis, the multi-site editing efficiency is further improved through the promoter replacement, the introduction and optimization of a synthetic spacing sequence in the crRNA array and the modification of a DR motif. Finally, the function of the MultiduBE is verified through the generation of a multi-resistance strain, the de novo generation of a riboflavin producing strain, and the increased yield of a surfactin producing strain.

A first object of the invention is to provide a multi-site dual-function base editor. The multi-site dual-function base editor comprises a plasmid comprising a cytosine deaminase, a uracil glycosylase inhibitor (UGI), a adenine deaminase TadA, a defective nuclease dCas12a, and a crRNA insertion region.

When the cytosine deaminase is hAPOBEC3A, the adenine deaminase TadA is located after hAPOBEC3A.

When the cytosine deaminase is hAID, the adenine deaminase TadA is located before hAID.

Further, the elements are arranged on the plasmid in any sequence of (1) the cytosine deaminase hAPOBEC3A, the adenine deaminase TadA, the defective nuclease dCas12a, and the uracil glycosylase inhibitor from front to back; and (2) the adenine deaminase TadA, the cytosine deaminase hAID, the defective nuclease dCas12a, and the uracil glycosylase inhibitor from front to back.

Further, the defective nuclease dCas12a has an amino acid sequence as shown in SEQ ID NO. 1.

Further, the cytosine deaminase hAPOBEC3A is deposited under GenBank Accession No. KM266646.1; the cytosine deaminase hAID is deposited under GenBank Accession No. AAM95402.1; and the adenine deaminase TadA has an amino acid sequence as shown in SEQ ID NO. 2.

Further, the crRNA insertion region comprises a crRNA array. In practical use, technicians can replace crRNA as needed to realize multi-site dual editing of different sequences.

Further, the crRNA array is constitutively expressed, and the cytosine deaminase, the uracil glycosylase inhibitor, the adenine deaminase TadA, and the defective nuclease dCas12a are inducibly expressed.

Further, the expression of the cytosine deaminase, the uracil glycosylase inhibitor, the adenine deaminase TadA and the defective nuclease dCas12a is regulated by a repressor LacI and a promotor P_{grac100}, or by a repressor TetR and a promoter Pₜₑₜ.

Further, the Pₜₑₜ sequence bearing the repressor TetR is as shown in SEQ ID NO. 5.

Further, the expression of the crRNA array is regulated by the promoter P_{veg}.

Further, any synthetic spacing sequence (Sp1-4) having a nucleotide sequence as shown in SEQ ID NOs. 6-9 is inserted in the crRNA array. Specifically, the synthetic spacing sequence is inserted between a DR motif and a spacer.

Further, the spacer has a length greater than 17 bp, preferably 17-26 bp, and further preferably 23 bp.

Further, the DR motif in the crRNA array is extended; and specifically, the extended DR motif has a nucleotide sequence as shown in SEQ ID NO. 11.

Further, the plasmid comprises a temperature-sensitive replicon.

Further, the temperature-sensitive replicon includes, but is not limited to, pE194ts.

A second object of the invention is to provide a fusion protein for multi-site dual-function base editing. The fusion protein comprises any one of
(1) a fusion sequence of the cytosine deaminase hAPOBEC3A, the adenine deaminase TadA, the defective nuclease dCas12a and a uracil glycosylase inhibitor in sequence; and
(2) a fusion sequence of the adenine deaminase TadA, the cytosine deaminase hAID, the defective nuclease dCas12a and a uracil glycosylase inhibitor in sequence.

Further, the fusion protein has an amino acid sequence as shown in SEQ ID NOs. 3-4.

A third object of the invention is to provide a recombinant strain comprising the multi-site dual-function base editor or the fusion protein.

Further, the recombinant strain is constructed with B. subtilis or E. coli as a starting strain.

A fourth object of the invention is to provide use of the multi-site dual-function base editor, the fusion protein or the recombinant strain in gene editing.

A fifth object of the invention is to provide use of the multi-site dual-function base editor, the fusion protein or the recombinant strain in the construction of a mutant.

A sixth object of the invention is to provide use of the multi-site dual-function base editor, the fusion protein or the recombinant strain in biological synthesis.

A seventh object of the invention is to provide use of the multi-site dual-function base editor, the fusion protein or the recombinant strain in metabolic regulation.

### BENEFICIAL EFFECTS OF THE INVENTION

According to the invention, a single-plasmid multi-site dual-function base editor (MultiduBE) based on CRISPR/Cas12a is designed and constructed, and base editing (C→T and A→G) at 5 sites of the genome is realized simultaneously under the guidance of a single crRNA array through the optimization and modification. According to the results of sequence alignment, related target genes are mutated, and the mutant strain develops resistance to tetracycline, rifampicin, spectinomycin and streptomycin simultaneously. In addition, 4 target genes associated with riboflavin synthesis are mutated, and a mutant strain that can produce 344.8 mg/L riboflavin is obtained by mutation from a wild-type strain that cannot produce riboflavin. The yield is 16 times the yield of a control strain where only the riboflavin synthesis gene cluster is strengthened. Finally, 5 target genes of a surfactin producing strain is mutated, and a mutant strain with 41.9% increased yield is obtained. In the multi-site dual-function base editor (MultiduBE), only a specific crRNA array is required to be constructed. Compared with the traditional genome editing method based on homology-directed repair, the construction process of a homologous repair template is saved. Compared with a dual-function base editor based on dCas9 or nCas9, the use of multiple promoters to express sgRNAs of different target genes is not required. The editor only consists of a temperature-sensitive plasmid, which can be eliminated by heat culture after the genome editing is completed. The mutant strain finally obtained only has base mutations at specific target sites on the genome (C→T and A→G, and G→A and T→C on the corresponding complementary strand), thus achieving the effect of multi-site efficient targeted mutagenesis.

### BRIEF DESCRIPTION OF THE DRAWINGS

FIG. 1 shows the working principle of a base editor;

FIG. 2 shows the design and analysis flow of a multi-site dual-function base editor (MultiduBE) based on CRISPR/Cas12a, where (a) a map of a plasmid backbone of a base editor; (b) two BsaI restriction sites are used for rapid assembly of a crRNA array; (c) a cytosine deaminase, an adenine deaminase, UGI, and dCas12a are coupled to construct multi-site dual-function base editor (MultiduBE); and (d) rapid analysis of base editing based on Sanger sequencing;

FIG. 3 shows the construction of a multi-site dual-function base editor (MultiduBE) based on CRISPR/Cas12a, where (a) 5 base editing verification target sites selected on genes aprE and nprE; (b) schematically shows the multi-site dual-function base editing mediated by a crRNA array, where the array is formed by a directed repeat (DR) sequence and a spacer sequence that are arranged alternately; and (c) the composition and structure optimization of a multi-site dual-function base editor (MultiduBE);

FIG. 4 shows the promoter replacement in a multi-site base editor;

FIG. 5 shows the optimization and modification of a crRNA array, where (a) a synthetic spacing (Sp) sequence is added in the crRNA array; (b) extended synthetic spacing sequence and DR sequence; (c) varying length of spacer in the crRNA array; and (d) optimized construction method of the CRRNA array;

FIG. 6 shows the influence of the optimization and modification of the crRNA array on the editing efficiency, where (a) the change of editing efficiency after adding different Sp sequences and changing the spacer; and (b) the influence of the Sp sequence on the processing of the crRNA array mediated by dCas12a;

FIGS. 7 and 8 show the analysis of mutants produced by using a multi-site dual-function base editor (MultiduBE), where FIG. 7 shows the analysis of a mutant produced by using pWLT-duBE-1a, and FIG. 8 shows the analysis of a mutant produced by using pWLT-duBE-2b, in which the data on the left and right is the ratios before and after adding Sp4, respectively, the asterisk represents the mutation in the complementary strand where the crRNA is located, and ~ represents a single base deletion.

FIG. 9 shows the effect of the dual-function base editor (MultiduBE) in E. coli;

FIG. 10 shows the use of the multi-site dual-function base editor (MultiduBE) in resistance development, where (a) alignment of related gene sequences; (b) edited target site for resistance development; (c) production of a dual-resistant mutant strain mediated by the multi-site dual-function base editor (MultiduBE); and (d) production of a tetra-resistant mutant strain mediated by the multi-site dual-function base editor (MultiduBE);

FIG. 11 shows the de novo production of a riboflavin producing strain mediated by the multi-site dual-function base editor (MultiduBE), where (A) riboflavin synthesis pathway in Bacillus subtilis; (b) a target site for targeted mutagenesis for riboflavin synthesis regulation; (c) screening of a strain highly producing riboflavin based on a microfluidic system; (d) a 96-well plate and fluorescence observation of a riboflavin synthesizing mutant strain; and (e) shake flask fermentation of a riboflavin synthesizing mutant strain; and

FIG. 12 shows the increase of surfactin production mediated by the multi-site dual-function base editor (MultiduBE), where (a) a target site for targeted regulation of surfactin; (b) targeted mutagenesis and screening of a surfactin producing mutant strain; and (c) shake flask fermentation of a surfactin producing mutant strain.

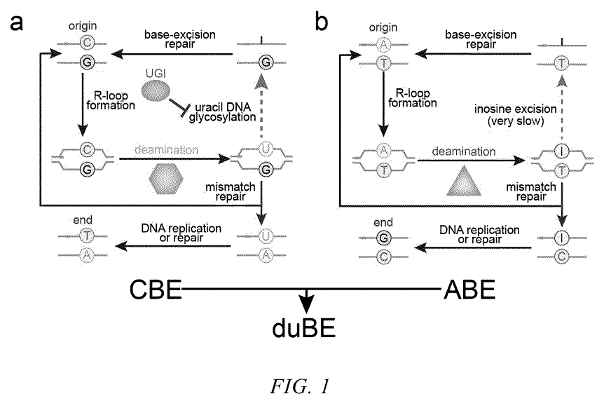

### DETAILED DESCRIPTION OF THE PREFERRED EMBODIMENTS

The invention will be further described below with reference to the accompanying drawings and specific examples, so that those skilled in the art can better understand and implement the invention; however, the invention is not limited thereto.

The materials and methods involved in the invention are as follows:

The PrimeSTAR high-fidelity DNA polymerase for amplifying the genomic fragment and the T4 DNA ligase for connecting the crRNA array are purchased from Takara Company. The Phanta Max Master Mix (Dye Plus) polymerase for amplifying the crRNA array fragments and the Taq DNA polymerase for colony PCR are purchased from Vazyme Biotech Co., Ltd. The restriction endonuclease BsaI for the construction of the crRNA array is purchased from NEB Company. The plasmid extraction kit is purchased from Sangon Biotech (Shanghai) Co., Ltd. The nucleic acid purification kit for PCR products is purchased from Thermo Scientific Company. The riboflavin and surfactin standards are purchased from Orileaf Bio-Technology Co., Ltd.

For the conventional cell culture, an LB medium is used, which contains: tryptone 10 g/L, yeast powder 5 g/L, and NaCl 10 g/L. In the medium, the final concentrations of kanamycin is 50 μg/mL, the final concentrations of tetracycline is 20 μg/mL, the final concentrations of rifampicin is 50 μg/mL, the final concentrations of spectinomycin is 50 μg/mL, the final concentrations of streptomycin is 200 μg/mL, the final concentrations of IPTG is 1 mM, and the final concentrations of anhydrotetracycline (aTC) is 500 nM.

When riboflavin and surfactin are produced by shake flask fermentation, the single colony activated by streaking is picked up into a 250 mL flat-bottomed shake flask with 20 mL LB medium, and a seed solution is prepared by incubation at 37° C. and 220 rpm for 12 h. Then, the seed solution is transferred to a 250 mL baffled shake flask with 50 ml fermentation medium at an inoculation amount of 5%, and fermented at 37° C. and 220 rpm. 1 mL is sampled every 12 h, to determine OD₆₀₀, and the glucose and product contents. The fermentation medium contains glucose 80 g/L, tryptone 6 g/L, yeast powder 12 g/L, urea 6 g/L, glycerol 5 g/L, K₂HPO₄·3H₂O 12.5 g/L, KH₂PO₄ 2.5 g/L, and Mg₂SO₄ 3 g/L.

Unless otherwise specified, the experimental method, detection method, and preparation method disclosed in the invention are all conventional molecular biological, biochemical, cell biological, and recombinant DNA technology in the art, as well as conventional techniques in related art. These techniques are well described in the existing literatures.

### Example 1: Design and Construction of Multi-Site Dual-Function Base Editor (MultiduBE) Based on CRISPR/Cas12a

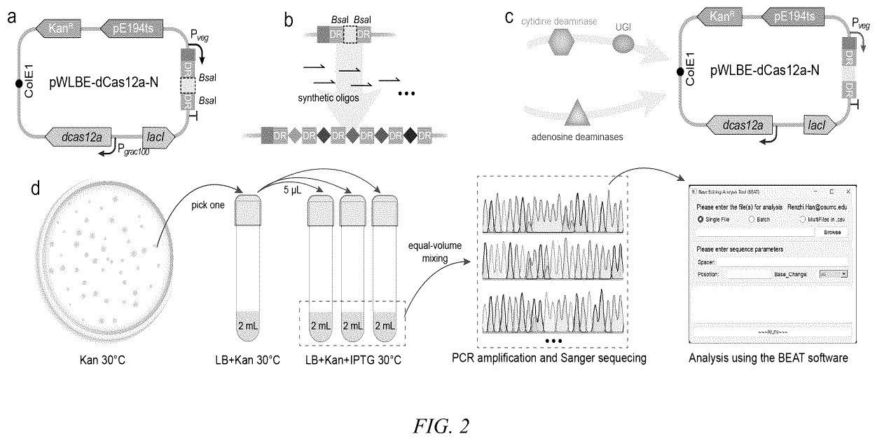

As shown in FIG. 2a, A base editing plasmid backbone pWLBE-dCas12a-N (MolecularCloud No.: MC_0101391) containing dCas12a (having an amino acid sequence as shown in SEQ ID NO. 1) was constructed, in which dCas12a was expressed by using the Bacillus subtilis IPTG inducible promoter P_{grac100}. The plasmid could be replicated in E. coli and Bacillus subtilis and screened with kanamycin. In addition, the backbone had the temperature-sensitive replicon pE194ts of Bacillus subtilis, which could be quickly removed from Bacillus subtilis when cultured at 50° C. In addition, an insertion region for a crRNA array was located downstream of the promoter P_{veg} which was active in both E. coli and Bacillus subtilis, and the region had two BsaI restriction sites. A multiplex crRNA array was rapidly assembled by a previously optimized synthetic oligos mediated assembly of crRNA array (SOMACA) method (Reference: Wu, Y., Liu, Y., Lv, X., Li, J., Du, G., Liu, L., 2020. CAMERS-B: CRISPR/Cpf1 assisted multiple-genes editing and regulation system for Bacillus subtilis. Biotechnology and Bioengineering 117, 1817-1825.) (FIG. 2b). Specifically, when a single crRNA was expressed, the crRNA could be directly obtained by annealing a pair of primers with an overlapping region (where the primer concentration was 10 uM, and a 20 uL system contained the upstream and downstream primers of each 10 uL; reaction conditions: 2 min at 98° C., cooling to 4° C. at 0.1° C./S, and holding at this temperature). The annealed product was diluted by 10 times, 1 uL was sampled, and the annealed product was connected to a vector cleaved by BsaI. When multiple crRNAs were designed to form a CRRNA array, multiple pairs of primers with an overlapping region were subjected to PCR (the primer concentration was 10 uM, and a 20 uL system contained 10 uL DNA polymerase, and the upstream and downstream primers of each 5 uL; and a standard PCR program was used, with an extension time of 5 sec and 10 cycles). After 10-time dilution, a double-stranded DNA with BsaI at two ends was obtained. Each 1 μL of the double-stranded DNA was subjected to golden gate with a plasmid. The product was transformed into E. coli, colony PCR was performed with one primer from the plasmid skeleton and one primer from crRNA, and single colonies with bands were picked up for sequencing to screen positive clones containing the required crRNA.

As shown in FIG. 2c, to construct a multi-site dual-function base editor (MultiduBE), a cytosine deaminase (hAPOBEC3A, hAID, LjCDA1L2_1, deposited under GenBank Accession Nos. KM266646.1, AAM95402.1, and MG495262.1), an adenine deaminase (TadA9, having an amino acid sequence as shown in SEQ ID NO. 2) and UGI (GenBank: YP_009283008.1) well matching dCas12a were respectively introduced into the plasmid backbone. Finally, plasmids containing a specific crRNA array and different fusion proteins were transformed into Bacillus subtilis, and quickly analyzed for the efficiency of base editing by using the operation steps shown in FIG. 2d, to screen a multi-site dual-function base editor (MultiduBE) with high activity. Specific operation steps were as follows: 1. The editing plasmid connected with a specific crRNA array was transformed into Bacillus subtilis. The cells were coated on a plate containing kanamycin, and cultured at 30° C. for 12 h. Then, 1 single colony was picked up and inoculated into a 14 mL shake flask containing 2 mL LB medium (containing kanamycin), and cultured at 30° C. for 12 h. Then, 5 μL of the cell suspension was transferred to three 14-mL shake flasks with 2 ml of LB medium (containing kanamycin and IPTG), and cultured with shaking at 30° C. for 36 h to induce the expression of the base editing system. Finally, 50 μL of each of the 3 parallel samples after the editing was completed were mixed in equal volume, and then 2 μL of the mixed cell suspension was added to 50 μL of a PCR reaction system to amplify the target gene fragment. The DNA fragment was purified, sanger sequencing was performed and the sequencing results were analyzed by BEAT software (https://hanlab.cc/beat/).

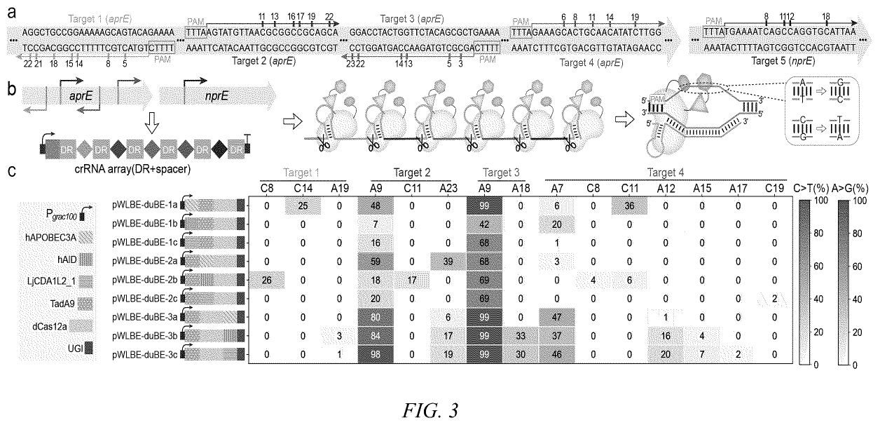

Here, editing verification target sites on aprE and nprE were selected and used for the construction and optimization of the multi-site dual-function base editor. As shown in FIG. 3a, 4 target sites on aprE gene and 1 target site on nprE gene were selected. The crRNAs targeting the 5 target sites were assembled by the SOMACA method described above to obtain a crRNA array. The array was cleavable by dCas12a to complete the guidance to the multiple sites. The dual-function base editing (C→T and A→G) of a non-targeted strand was completed under the action of the cytosine deaminase and adenine deaminase fused to dCas12a (FIG. 3b).

As shown in FIG. 3c, the composition and structure of the multi-site dual-function base editor (MultiduBE) were optimized. 3 cytosine deaminases (hAPOBEC3A, hAID, and LjCDA1L2_1), 1 adenine deaminase (TadA9), UGI and dCas12a were combined and arranged on the pWLBE-dCas12a-N backbone to construct various fusion proteins. Subsequently, a crRNA array targeting 5 target sites on aprE and nprE genes was constructed. The method described in FIG. 2d was used for multi-site base editing analysis and verification (see Table 1 for primer sequences for amplification and sequencing of aprE and nprE). It can be seen that pWLBE-duBE-1a and pWLBE-duBE-2b have a high multi-site dual-function editing function, and both can realize the dual-function editing of 4 target sites (C→T and A→G). The amino acid sequences of the fusion proteins duBE-1a and duBE-2b are shown in SEQ ID NO.3 and SEQ ID NO.4. Although pWLBE-duBE-3b and pWLBE-duBE-3c can edit 4 sites, they only have adenine editing activity (A→G). The number of editable sites by fusion proteins having other compositions is less than 4. Finally, duBE-1a having an amino acid sequence as shown in SEQ ID NO. 3 and duBE-2b having an amino acid sequence as shown in SEQ ID NO. 4 are used as multi-site dual-function base editors (MultiduBE), and further optimized to improve their editing efficiency.

TABLE 1
Primer sequences for amplification
and sequencing of aprE and nprE

| Use of primer | Primer sequence |
|---|---|
| Upstream primer for | AATAAGCGGATACTC |
| amplifying aprE gene | TCTGACG |
| Downstream primer for | CTGTTAAAACGTTTC |
| amplifying aprE gene | AGGATTTGGC |
| Upstream primer for | GCATTCCAGGCTGCT |
| amplifying nprE gene | TCAACTT |
| Downstream primer for | GCATTCCAGGCTGCT |
| amplifying nprE gene | TCAACTT |
| Upstream primer for | CATTTCAGCATAATG |
| aprE sequencing | AACATTTACTCATGT |
| | C |
| Downstream primer for | AGATTTTCAGAGGCA |
| aprE sequencing | GCCAT |
| Primer for nprE | GAATGAGAGCTGCCT |
| sequencing | TGGCA |

### Example 2: Optimization and Modification of Multi-Site Dual-Function Base Editor (MultiduBE)

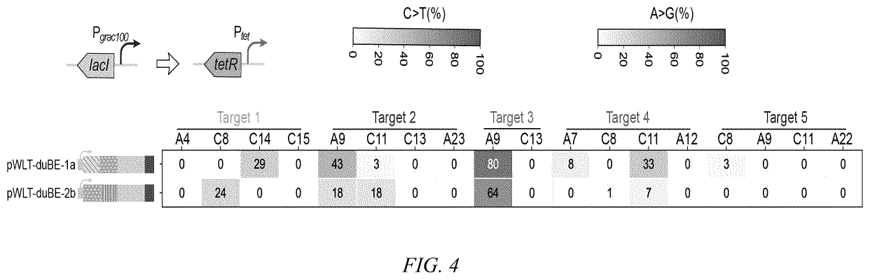

Under the guidance of the crRNA array targeting 5 sites, pWLBE-duBE-1a and pWLBE-duBE-2b can only realize the dual-function base editing of 4 sites. Therefore, further by the promoter replacement and the modification of the structure of the crRNA array, the multi-site dual-function base editor (MultiduBE) is optimized and modified, so as to further improve the editing efficiency. As shown in FIG. 4, the original P_{grac100} promoter in pWBE-duBE-1a and pWBE-duBE-2b was replaced by the tetracycline-inducible promoter Pₜₑₜ (corresponding repressor LacI was replaced by TER, and the Pₜₑₜ sequence bearing the repressor TetR was as shown in SEQ ID NO. 5). The plasmids pWLT-duBE-1a and pWLT-duBE-2b were obtained. pWLT-duBE-1a can realize the editing of 5 sites, which enlarges the editing window compared with the case before promoter modification. However, the editing efficiency of the 5th site is only 3%. However, pWLT-duBE-2b can still only edit 4 sites, and the editing efficiency of the 4th site is less than 10%.

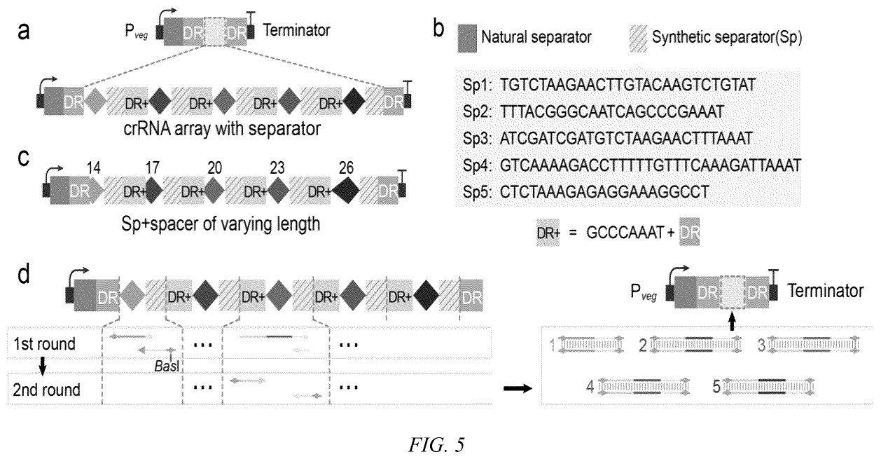

To further enhance the editing efficiency of the multi-site dual-function base editor (MultiduBE), an attempt was made to add a synthetic spacing (Sp) sequence to the crRNA array. As shown in FIGS. 5a and 5b, a different Sp sequence (the nucleotide sequences of Sp 1-5 were as shown in SEQ ID NOs. 6-10) was introduced between DR and the spacer, to further improve the editing efficiency. In addition, to optimize the construction process of the crRNA array, the original DR sequence was extended to increase its Tm value to about 55° C. (the nucleotide sequence of DR+ was as shown in SEQ ID NO. 11). Moreover, the length of the spacer in the crRNA array was changed to investigate its influence on the editing efficiency (FIG. 5c).

As shown in FIG. 5d, the specific steps to optimize the construction method of the crRNA array after adding the Sp sequence were as follows: (1) The 1st crRNA was amplified into a double-stranded product by one round of PCR directly using a pair of primers. The amplification system contained 10 μL Phanta Max and 5 μL of each of the upstream and downstream primers. 30 cycles of amplification were performed according to the instructions. (2) The following crRNAs were amplified by one rounds of PCR. In the first round, PCR was performed for the formation of a double-stranded product (the upstream primer was single-stranded DR+, spacer and Sp sequence, and the downstream primer was a complementary single-stranded Sp sequence), to obtain a double-stranded DNA fragment with repeated sequences (DR+ and Sp sequence) at both ends. The amplification system contained 10 μL Phanta Max and 5 μL of each of the upstream and downstream primers. 15 cycles of amplification were performed according to the instructions. In the second round of PCR, a sticky end was added to both ends of the double-stranded DNA fragment obtained in the first round for golden gate assembly. The 3′ end of the upstream and downstream primers used would bind to the repeated sequence, and thus be universal. The 5′ end was adjusted according to the enzymatic cleavage interface needed to be added (refer to the SOMACA method). The amplification system contained 10 μL Phanta Max, 5 μL of each of the upstream and downstream primers, and 1 μL PCR product obtained in the first round. 30 cycles were performed. (3) The amplified crRNA double-stranded DNA fragments were diluted by ten times and mixed. A golden gate assembly system (including, in every L, T4 DNA ligase 2 μL, BasI 1 μL, T4 DNA ligase buffer 1 μL, plasmid 1 μL, crRNA mix 2 μL, and ddH₂O 3 μL). The fragments were assembled through the procedures in Table 2, then transformed into E. coli, and sequenced. After the construction was confirmed to be successful, the construct was transformed into Bacillus subtilis for genome editing verification.

TABLE 2
Assembly procedure of crRNA array

| Cycling | Temperature (° C.) | Time (min) |
|---|---|---|
| 60 | 37 | 4 |
| | 16 | 5 |
| 1 | 37 | 60 |
| 1 | 50 | 5 |
| 1 | 80 | 5 |
| 1 | 12 | ∞ |

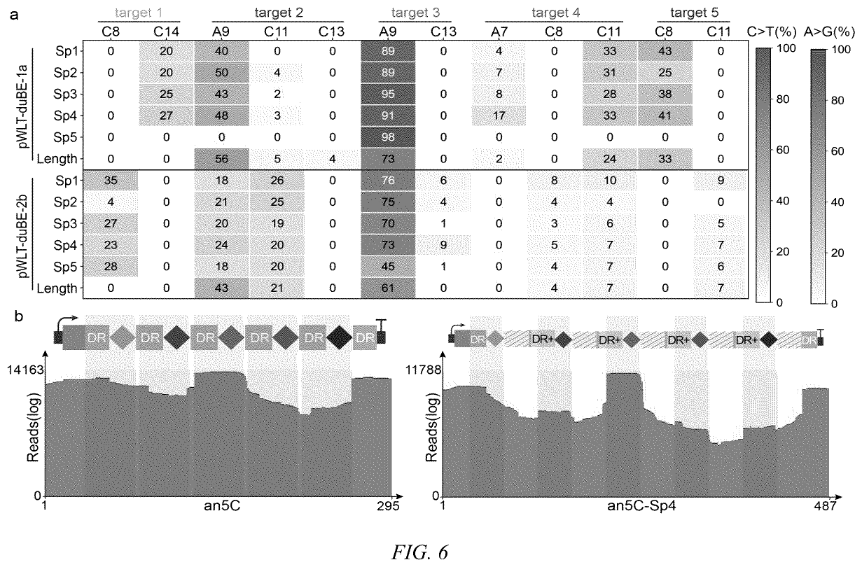

As shown in FIG. 6a, Sp4 (having a nucleotide sequence as shown in SEQ ID NO. 9) has obvious improvement effect on the editing efficiency of pWLT-duBE-1a and pWLT-duBE-2b. Under the guidance of the crRNA array containing Sp4, they can both realize the dual-function base editing of 5 sites. Sp 1-3 also have a certain improvement effect on the editing efficiency. In addition, changing the length of the spacer will also have a certain impact on the editing efficiency. When the length is shortened to 14 bp, dCas12a cannot be guided to edit the genome. Spacers larger than 17 bp have a guidance function, but the spacer sequence of 23 bp is still the most favorable for editing. In addition, the microRNA produced by the expression of the crRNA array before and after adding Sp4 is sequenced, and it is found that the addition of Sp4 really promotes the processing and cleavage of the crRNA array, thus improving the multi-site targeting editing efficiency (see FIG. 6b).

### Example 3: Analysis of Mutant Produced by Using Multi-Site Dual-Function Base Editor (MultiduBE)

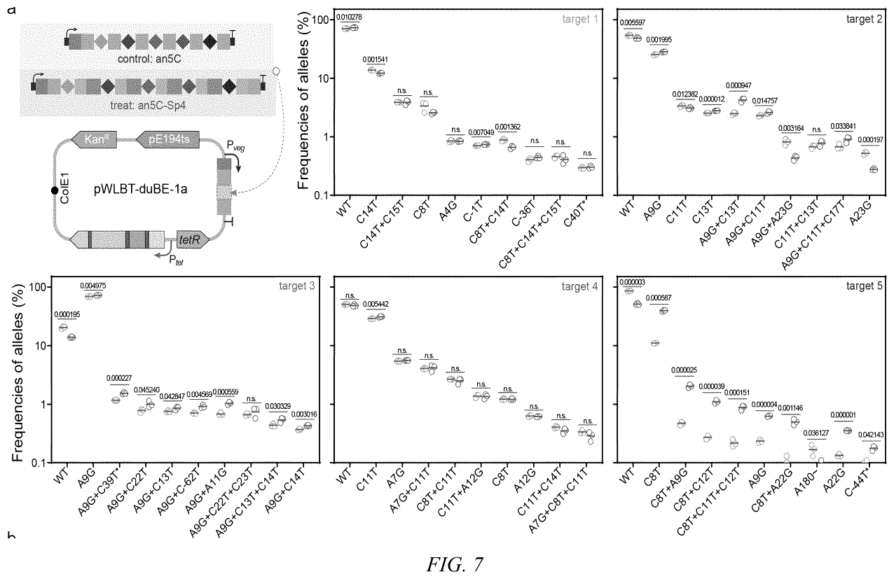

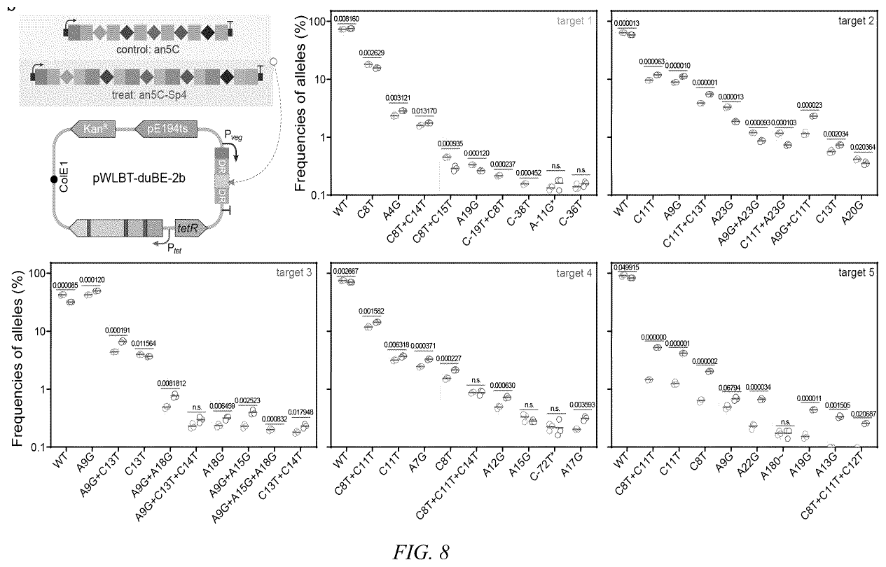

In the above modification and optimization process, the results of base editing are all analyzed by Sanger sequencing. The analysis process is short in time and low in price, so it is suitable for the initial construction and optimization of the multi-site dual-function base editor (MultiduBE). However, the accuracy of Sanger sequencing is low, low-frequency mutations cannot be found, only the approximate proportion of base mutations can be obtained, the specific composition of the mutant cannot be obtained, and even whether the dual-function base editing (C→T and A→G) occurs in the same sequence cannot be determined. Therefore, the multi-site base editing by pWLT-duBE-1a and pWLT-duBE-2b under the guidance of the crRNA array before and after adding Sp4 was further analyzed through high-throughput sequencing. Target sites edited in the aprE and nprE genes in three parallel samples after editing were respectively amplified and sequenced, and the composition of 9 mutants with the highest frequency of occurrence was analyzed. As shown in FIGS. 7-8, the compositions of the mutants produced by using pWLT-duBE-1a and pWLT-duBE-2b are quite different. For both of them, the introduction of Sp4 does not improve the editing efficiency at target site 1, and the editing efficiency decreases slightly after the addition of Sp4. This may be because the addition of Sp4 promotes the maturation of the following crRNA, thus reducing the relative proportion of dCas12a bound to the first crRNA. For the target site 5 having a low editing efficiency, adding Sp4 can significantly improve the editing efficiency.

As shown in FIG. 7, when pWLT-duBE-1a is used for multi-site base editing, the editing efficiency of A→G at target site 1 is low, 8 of the 9 mutants with the highest frequency of occurrence are of single-site or multi-site C→T mutations, and no mutants with both C→T and A→G are found. With respect to the following target sites in the gene, mutants with both C→T and A→G are observed. Low-frequency (less than 2%) bystander editing out-of-protospacer (BEOP) at other sites than the crRNA targeting site is also found at target sites 1, 3 and 5. For example, the complementary strand to the position 40 of target site 1 has a mutation of C→T (0.31 and 0.32% before and after adding Sp4, respectively). As shown in FIG. 8, when pWLT-duBE-2b is used for multi-site base editing, the editing efficiency of A→G at target site 1 is higher than that of pWLT-duBE-1a, but mutants with both C→T and A→G are still not observed. Unlike pWLT-duBE-1a, the mutant with C→T and A→G is found only at target site 2 and target site 3, and low-frequency (less than 0.5%) BEOP is found at target sites 1, 4 and 5.

### Example 4: Use of Multi-Site Dual-Function Base Editor (MultiduBE) in E. coli

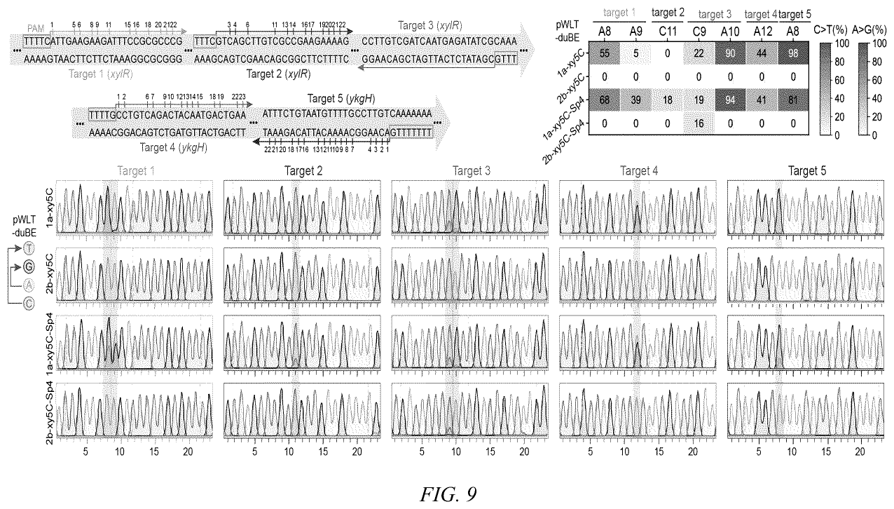

The promoter Pₜₑₜ for expressing dCas12a and the promoter P_{veg} for expressing crRNA in pWLT-duBE-1a and pWLT-duBE-2b are both active in E. coli. Accordingly, whether the multi-site dual-function base editor (MultiduBE) can function in E. coli and Bacillus subtilis is verified. As shown in FIG. 9, 5 target sites on the genes xylR and ykgH from E. coli were selected, and a crRNA array was constructed. After sequencing, it is found that pWLT-duBE-1a has multi-site dual-function base editing activity in E. coli, and the editing target sites can be increased from 4 to 5 after adding Sp4.

### Example 5: Use of Multi-Site Dual-Function Base Editor (MultiduBE) in Resistance Development

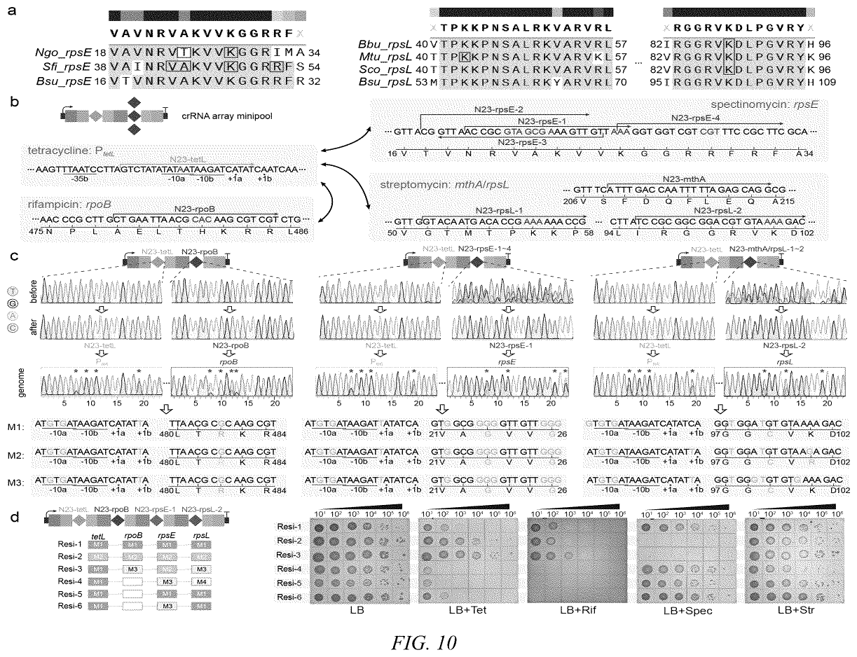

The function of the multi-site dual-function base editor (MultiduBE) was verified by forming multiple resistant mutants. As shown in FIG. 10a, the antibiotic target gene rpsE (encoding the ribosomal protein S5) in the spectinomycin-resistant mutants of Neisseria gonorrhoeae and Streptomyces roseosporus was mutated in the previous report. The sequence of the RpsE protein in the above two hosts was aligned with the sequence of the RpsE protein in Bacillus subtilis. The potential to-be-mutated target site for producing spectinomycin resistance was determined in Bacillus subtilis. The streptomycin resistance could be produced by mutating, in Borrelia burgdorferi, Mycobacterium tuberculosis and Streptomyces coelicolor, the rpsL gene (encoding the ribosomal protein S5). The sequence of the RpsL protein in the above hosts was aligned with the sequence of the RpsL protein in Bacillus subtilis. The potential to-be-mutated target site for producing streptomycin resistance was determined in Bacillus subtilis. According to previous reports on genes in Bacillus subtilis, the tetracycline resistance could be produced by mutating the promoter region of the tetL gene (tetracycline efflux protein leader peptide), the rifampicin resistance could be produced by mutating the rpoB gene (encoding the R subunit of the RNA polymerase), and the streptomycin resistance could also be produced by mutating the mthA gene (encoding methylthioadenosine/s-adenosylhomocysteine nucleosidase).

Here, crRNAs targeting P_{tetL} and rpoB were constructed into a binary array, to investigate whether it could guide the multi-site dual-function base editor (MultiduBE) to perform targeted mutagenesis on the genome and, produce tetracycline and rifampicin resistance; In addition, 4 different crRNAs were set for the potential mutation sites in rpsE, which were combined with the crRNAs targeting P_{tetL} to form four binary array, to produce tetracycline and spectinomycin resistance. In addition, two different crRNAs were designed for the rpsL gene and one crRNA was designed for mthA, which was combined with the crRNA targeting P_{tetL} to form three binary array, to produce tetracycline and streptomycin resistance. Because the product spectra of the two multi-site dual-function base editors (MultiduBE) pWLT-duBE-1a and pWLT-duBE-2b are quite different, more mutants can be produced by using them in mixture. Therefore, the binary crRNA array library was simultaneously ligated to a mixture of pWLT-duBE-1a and pWLT-duBE-2b, and then transformed into Bacillus subtilis strain BSZRG (previously constructed based on Bacillus subtilis 168, Reference: Li, Y., Wu, Y., Liu, Y., Li, J., Du, G., Lv, X., Liu, L., 2022. A genetic toolkit for efficient production of secretory protein in Bacillus subtilis. Bioresource Technology 363, 127885.) targeted induced mutation. As shown in FIG. 10c, the binary crRNA array containing pWLT-duBE-1a and pWLT-duBE-2b were transformed into Bacillus subtilis for induced mutation, and then corresponding antibiotics were added for screening. The screened mixed bacterial cell suspension was subjected to sequencing of target genes. Finally, three single colonies were picked up from each resistant combination for sequencing of target genes after streaking and separation. Before the resistance screening, the sequencing upon the use of the crRNA array to produce tetracycline+rifampicin resistance shows a single peak pattern; and the sequencing upon the use of the crRNA arrays to produce tetracycline+spectinomycin resistance and tetracycline+streptomycin resistance shows a superimposed peak pattern because of the multiple combinatorial sequencing. After adding the corresponding double antibodies for screening, the sequencing upon the use of the original mixed crRNA array also changes to a single peak pattern, indicating that the targeting crRNA that can produce corresponding resistance is screened and enriched in this process. Through the sequencing of corresponding target genes on the genome of the screened mixed bacterial cell suspension, it is found that the mutated sites are consistent with the finally enriched crRNA array, which shows that the crRNA array can be used as a tracker for mutated targets on the genome. Sequencing of three single colonies of three combinations of dual resistance shows that three single colonies of tetracycline+rifampicin and tetracycline+spectinomycin resistant mutants all have the same mutations, and three single colonies of tetracycline+streptavidin resistant mutants have different mutations.

On this basis, by the enrichment process of crRNA arrays in the screening of resistant mutants, the best crRNAs corresponding to four kinds of resistance were determined, which were constructed into a quaternary crRNA array (having a nucleotide sequence as shown in Sequence 12), for targeted mutagenesis of a strain with four mutations. By using the crRNA array, one tetracycline+rifampicin+spectinomycin+streptomycin resistant mutant strain is obtained by mutagenesis, two tetracycline+rifampicin+streptomycin resistant mutant strains are obtained by mutagenesis, and three tetracycline+spectinomycin+streptomycin resistant mutant strains are obtained by mutagenesis (FIG. 10d).

### Example 6: De Novo Production of a Riboflavin Producing Strain Mediated by the Multi-Site Dual-Function Base Editor (MultiduBE)

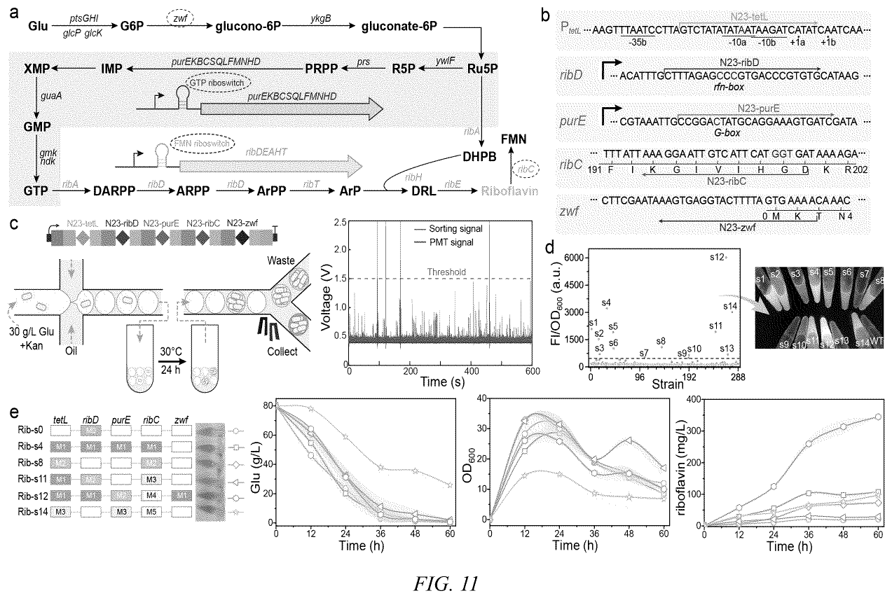

Riboflavin (also called vitamin B2) is a heat-resistant water-soluble vitamin essential in human body and a component of prosthetic group of flavoenzymes in the body. Its neutral solutions in water and ethanol are yellow and strongly fluoresce. As shown in FIG. 11a, there are two riboswitches (guanine riboswitch and FMN riboswitch) in the riboflavin synthesis pathway in Bacillus subtilis, which will produce feedback inhibition on the synthesis pathway. In addition, the riboflavin kinase encoded by the essential gene ribC can convert riboflavin into FMN, This process consumes riboflavin, and the produced FMN further strengthens the feedback inhibition by the FMN riboswitch. In addition, 6-phosphogluconate dehydrogenase encoded by zwf, as a key step of intracellular carbon flow distribution in the pentose phosphate pathway, will also affect the riboflavin synthesis. Therefore, the guanine riboswitch and FMN riboswitch, the ribC gene and zwf gene were used as target sites for targeted mutagenesis in riboflavin synthesis. Moreover, the crRNAPₜₑₜ was also used to observe the base editing induced in the absence of a corresponding selection stress (FIG. 11b). Then the crRNAs acting on the above target sites were constructed into a five-element crRNA array (having a nucleotide sequence as shown in Sequence 13). As shown in FIG. 11c, a base editing plasmid containing the crRNA array was transformed into Bacillus subtilis strain G600 not producing riboflavin (previously constructed based on Bacillus subtilis 168, Reference: Li, Y., Wu, Y., Liu, Y., Li, J., Du, G., Lv, X., Liu, L., 2022. A genetic toolkit for efficient production of secretory protein in Bacillus subtilis. Bioresource Technology 363, 127885.), and 5 corresponding sites were subjected to targeted mutagenesis, to obtain a riboflavin synthesizing mutant strain by de novo production. The mutant library after mutagenesis was diluted to OD₆₀₀=0.1 with a culture medium containing 30 g/L glucose and kanamycin, wrapped in a single-cell droplet, and cultured at 30° C. for 24 h. Then the droplets with high fluorescence were sorted by a microfluidic system (riboflavin was itself fluorescent, and the output was proportional to the fluorescence intensity after it was secreted out of the cell). The cells in the collected droplets were coated on a plate containing kanamycin and cultured. Single colonies were picked up into a 96-well plate, followed by fluorescence determination and re-screening. A total of 12 mutant strains s1-s12 were obtained (FIG. 11d). Although tetracycline was not used for screening, the Pₜₑₜ site in these 12 strains was mutated. As shown in FIG. 11e, five mutant strains Rib-s4, Rib-s8, Rib-s11, Rib-s12 and Rib-s14 were selected. The base editing plasmids were removed and then the strains were fermented in a shake flask. A Rib-s0 strain was constructed as a control (where the P_{veg} promoter was used to replace the promoter and FMN riboswitch region of rib operon). The yield from shake flask fermentation of the five mutant strains is higher than that of the control strain Rib-s0 (21.5 mg/L), and the yield of the mutant strain Rib-s12 with the highest yield (344.8 mg/L) is 16 times that of the control strain.

### Example 7: Increase of Surfactin Production Mediated by the Multi-Site Dual-Function Base Editor (MultiduBE)

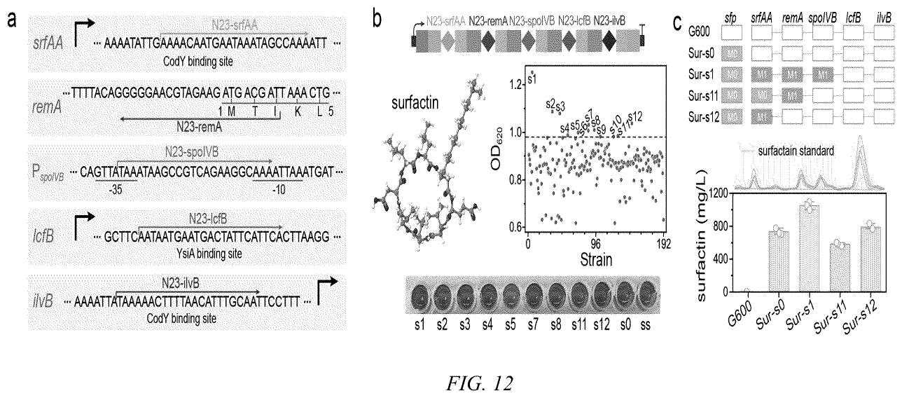

Surfactin is a lipopeptide biosurfactant synthesized by Bacillus-derived non-ribosomal peptide synthetase (NRPS), Compared with chemical surfactants, it has the advantages of anti-adhesion, anti-biofilm formation, anti-bacterial and anti-inflammatory, anti-mycoplasma, and anti-viral effects, and has a wide application prospect in biopharmaceuticals, environmental restoration, oil field exploitation, cosmetics and daily necessities. As shown in FIG. 12a, according to the related reports in the literatures, five genes including srfAA (encoding surfactin synthase), remA (encoding a regulatory protein of a gene related to biofilm formation), spoIVB (encoding serine protease), lcfB (encoding) and ilvB (encoding) were selected as editing target sites for surfactin synthesis regulation. Then five crRNAs acting on the above target sites were constructed into a five-element crRNA array (having a nucleotide sequence as shown in Sequence 14). The coding gene sfp of 4-phosphopantetheinyl transferase (for activating the surfactin synthase) in the wild-type Bacillus subtilis strain 168 is inactive, so it cannot be used to synthesize surfactin. Here, The sfp gene in Bacillus subtilis strain G600 was back mutated to restore its activity (the amino acid sequence is as shown in Sequence 15 after back mutation), and the strain Sur-S0 was obtained. As shown in FIG. 12b, the crRNA array containing a related gene targeting surfactin synthesis was connected to the base editing plasmid, and then transformed into strain Sur-s0 for targeted mutagenesis of the genes related to surfactin synthesis. The mutant strains were screened by chromogenic method (Reference: Yang, H., Yu, H., Shen, Z., 2015. A novel high-throughput and quantitative method based on visible color shifts for screening Bacillus subtilis THY-15 for surfactin production. Journal of Industrial Microbiology and Biotechnology 42, 1139-1147). The mutant strains Sur-s1 to Sur-s12 were obtained (where strains Sur-s6, Sur-s9 and Sur-s10 are wild-type or copies of other mutant strains). Finally, five strains Rib-G600, Sur-s0, Sur-s1, Sur-s11 and Sur-s12 were used for shake-flask fermentation. No surfactin is detected in the fermentation liquid of G600, surfactin of 702.7 mg/L can be synthesized by the control strain Sur-s0, and the yield of the mutant strain Sur-s1 is increased by 42.0% to 997.5 mg/L compared with the control strain (FIG. 12c).

Apparently, the above-described embodiments are merely examples provided for clarity of description, and are not intended to limit the implementations of the invention. Other variations or changes can be made by those skilled in the art based on the above description. The embodiments are not exhaustive herein. Obvious variations or changes derived therefrom also fall within the protection scope of the invention.

1. A multi-site dual-function base editor, the multi-site dual-function base editor comprising a plasmid comprising a cytosine deaminase, a uracil glycosylase inhibitor, a adenine deaminase TadA, a defective nuclease dCas12a, and a crRNA insertion region, wherein
when the cytosine deaminase is hAPOBEC3A, the adenine deaminase TadA is located after hAPOBEC3A; and
when the cytosine deaminase is hAID, the adenine deaminase TadA is located before hAID.

2. The multi-site dual-function base editor according to claim 1, wherein the elements are arranged on the plasmid in any sequence of (1) the cytosine deaminase hAPOBEC3A, the adenine deaminase TadA, the defective nuclease dcas12a, and the uracil glycosylase inhibitor from 5′ end to 3′ end; and (2) the adenine deaminase TadA, the cytosine deaminase hAID, the defective nuclease dCas12a, and the uracil glycosylase inhibitor from 5′ end to 3′ end.

3. The multi-site dual-function base editor according to claim 1, wherein the crRNA insertion region comprises a crRNA array.

4. The multi-site dual-function base editor according to claim 3, wherein the crRNA array is constitutively expressed, and the cytosine deaminase, the uracil glycosylase inhibitor, the adenine deaminase TadA, and the defective nuclease dCas12a are inducibly expressed.

5. The multi-site dual-function base editor according to claim 4, wherein the expression of the cytosine deaminase, the uracil glycosylase inhibitor, the adenine deaminase TadA and the defective nuclease dCas12a is regulated by a repressor LacI and a promotor P_{grac100}, or by a repressor TetR and a promoter Pₜₑₜ.

6. The multi-site dual-function base editor according to claim 3, wherein the expression of the crRNA array is regulated by the promoter P_{veg}.

7. The multi-site dual-function base editor according to claim 3, wherein a spacing sequence having any nucleotide sequence as shown in SEQ ID NOs. 6-9 is inserted in the crRNA array.

8. The multi-site dual-function base editor according to claim 7, wherein the spacing sequence is inserted between a DR motif and a spacer; and the spacer has a length greater than 17 bp.

9. The multi-site dual-function base editor according to claim 3, wherein the DR motif in the crRNA array is extended; and the extended DR motif has a nucleotide sequence as shown in SEQ ID NO. 11.

10. The multi-site dual-function base editor according to claim 1, wherein the plasmid comprises a temperature-sensitive replicon.

11. A fusion protein for multi-site dual-function base editing, comprising any one of
(1) a fusion sequence of the cytosine deaminase hAPOBEC3A, the adenine deaminase TadA, the defective nuclease dCas12a and a uracil glycosylase inhibitor in sequence; and
(2) a fusion sequence of the adenine deaminase TadA, the cytosine deaminase hAID, the defective nuclease dCas12a and a uracil glycosylase inhibitor in sequence.

12. A recombinant strain comprising the multi-site dual-function base editor according to claim 1.

13. The recombinant strain according to claim 12, wherein the recombinant strain is constructed with B. subtilis or E. coli as a starting strain.

14. Use of the multi-site dual-function base editor according to claim 1 in gene editing.

15. Use of the multi-site dual-function base editor according to claim 1 in the construction of a mutant.

16. Use of the multi-site dual-function base editor according to claim 1 in biological synthesis.

17. Use of the multi-site dual-function base editor according to claim 1 in metabolic regulation.


SEQUENCE LISTING (15 sequences)

SEQ ID NO: 1 - protein, 1300 aa, synthetic construct
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 2 - protein, 167 aa, synthetic construct
MSEVEFSHEYWMRHALTLAKRARDEREVPVGAVLVLNNRVIGEGWNRAIGLHDPTAHAEIMALRQGGLVMQNYRLIDATLYSTFEPCVMCAGAMIHSRIGRVVFGVRNSKRGAAGSLMNVLNYPGMNHRVEITEGILADECAALLCDFYRMPRRVFNAQKKAQSSIN

SEQ ID NO: 3 - protein, 1820 aa, synthetic construct
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESMSEVEFSHEYWMRHALTLAKRARDEREVPVGAVLVLNNRVIGEGWNRAIGLHDPTAHAEIMALRQGGLVMQNYRLIDATLYSTFEPCVMCAGAMIHSRIGRVVFGVRNSKRGAAGSLMNVLNYPGMNHRVEITEGILADECAALLCDFYRMPRRVFNAQKKAQSSINSGGSSGGSSGSETPGTSESATPESSGGSSGGSSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNNGSPKKKRKVSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 4 - protein, 1816 aa, synthetic construct
MSEVEFSHEYWMRHALTLAKRARDEREVPVGAVLVLNNRVIGEGWNRAIGLHDPTAHAEIMALRQGGLVMQNYRLIDATLYSTFEPCVMCAGAMIHSRIGRVVFGVRNSKRGAAGSLMNVLNYPGMNHRVEITEGILADECAALLCDFYRMPRRVFNAQKKAQSSINSGGSSGGSSGSETPGTSESATPESSGGSSGGSMDSLLMNRREFLYQFKNVRWAKGRRETYLCYVVKRRDSATSFSLDFGYLRNKNGCHVELLFLRYISDWDLDPGRCYRVTWFISWSPCYDCARHVADFLRGNPNLSLRIFTARLYFCEDRKAEPEGLRRLHRAGVQIAIMTFKDYFYCWNTFVENHGRTFKAWEGLHENSVRLSRQLRRILLPLYEVDDLRDAFRTSGSETPGTSESATPESSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNNGSPKKKRKVSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 5 - DNA, 837 bp, synthetic construct
  source 1..837: mol_type = other DNA
ttaggacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaataggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctggacatgtgattcctccttatttatgtagttactatataaaagcattagtgtatcaattccacgattttttctgaaatcctgctcgtttttaaaatatttttctagaatgaaaggggattttatgcttcagaacgctcggttgccgccgggcgttttttatactagtcaaaaactaaaaaaaatattgacactctatcattgatagagtataattaacg

SEQ ID NO: 6 - DNA, 27 bp, synthetic construct
  source 1..27: mol_type = other DNA
tgtctaagaacttgtacaagtctgtat

SEQ ID NO: 7 - DNA, 23 bp, synthetic construct
  source 1..23: mol_type = other DNA
tttacgggcaatcagcccgaaat

SEQ ID NO: 8 - DNA, 27 bp, synthetic construct
  source 1..27: mol_type = other DNA
atcgatcgatgtctaagaactttaaat

SEQ ID NO: 9 - DNA, 32 bp, synthetic construct
  source 1..32: mol_type = other DNA
gtcaaaagacctttttgtttcaaagattaaat

SEQ ID NO: 10 - DNA, 21 bp, synthetic construct
  source 1..21: mol_type = other DNA
ctctaaagagaggaaaggcct

SEQ ID NO: 11 - DNA, 27 bp, synthetic construct
  source 1..27: mol_type = other DNA
gcccaaataatttctactgttgtagat

SEQ ID NO: 12 - DNA, 368 bp, synthetic construct
  source 1..368: mol_type = other DNA
gtctaagaactttaaataatttctactgttgtagatgtctatatataataagatcatatgtcaaaagacctttttgtttcaaagattaaataggtgcccaaataatttctactgttgtagatctgaattaacgcacaagcgtcgtgtcaaaagacctttttgtttcaaagattaaatccacgcccaaataatttctactgttgtagataccgcgtagcgaaagttgttaaagtcaaaagacctttttgtttcaaagattaaatggtagcccaaataatttctactgttgtagattccgcggcggacgtgtaaaagacgtcaaaagacctttttgtttcaaagattaaataatttctactgttgtagat

SEQ ID NO: 13 - DNA, 454 bp, synthetic construct
  source 1..454: mol_type = other DNA
gtctaagaactttaaataatttctactgttgtagatgtctatatataataagatcatatgtcaaaagacctttttgtttcaaagattaaataggtgcccaaataatttctactgttgtagatctttagagcccgtgacccgtgtggtcaaaagacctttttgtttcaaagattaaatccacgcccaaataatttctactgttgtagatccggactatgcaggaaagtgatcgtcaaaagacctttttgtttcaaagattaaatggtagcccaaataatttctactgttgtagattcaccatgaatgacaattcctttgtcaaaagacctttttgtttcaaagattaaatccgagcccaaataatttctactgttgtagatttttcactaaaagtacctcacttgtcaaaagacctttttgtttcaaagattaaataatttctactgttgtagat

SEQ ID NO: 14 - DNA, 454 bp, synthetic construct
  source 1..454: mol_type = other DNA
gtctaagaactttaaataatttctactgttgtagataaaacaatgaataaatagccaaagtcaaaagacctttttgtttcaaagattaaataggtgcccaaataatttctactgttgtagatatcgtcatcttctacgttcccccgtcaaaagacctttttgtttcaaagattaaatccacgcccaaataatttctactgttgtagattaaataagccgtcagaaggcaaagtcaaaagacctttttgtttcaaagattaaatggtagcccaaataatttctactgttgtagataataatgaatgactattcattcagtcaaaagacctttttgtttcaaagattaaatccgagcccaaataatttctactgttgtagattaaaaacttttaacatttgcaatgtcaaaagacctttttgtttcaaagattaaataatttctactgttgtagat

SEQ ID NO: 15 - protein, 224 aa, synthetic construct
MKIYGIYMDRPLSQEENERFMSFISPEKREKCRRFYHKEDAHRTLLGDVLVRSVISRQYQLDKSDIRFSTQEYGKPCIPDLPDAHFNISHSGRWVICAFDSQPIGIDIEKTKPISLEIAKRFFSKTEYSDLLAKDKDEQTDYFYHLWSMKESFIKQEGKGLSLPLDSFSVRLHQDGQVSIELPDSHSPCYIKTYEVDPGYKMAVCAAHPDFPEDITMVSYEELL